(19) United States
(12) Patent Application Publication
(10) Pub. No.: US 2003/0175220 A1
(43) Pub. Date: Sep. 18, 2003

(54) Effect of electrolyzed solutions on acidogenesis of plaque

(76) Inventors: Xiao Bing Wang, Lutherville, MD (US); Shinkatsu Morisawa, Kyoto (JP)

Correspondence Address: TROUTMAN SANDERS LLP, BANK OF AMERICA PLAZA, SUITE 5200, 600 PEACHTREE STREET , NE, ATLANTA, GA, 30308-2216, US

(21) Appl. No.: 10/316,269

(22) Filed: Dec. 11, 2002

Related U.S. Application Data
(60) Provisional application No. 60/340,769, filed on Dec. 11, 2001.

(51) Int. Cl. A61K 7/20

(52) U.S. Cl. 424/53

(57) ABSTRACT

The present invention provides compositions and methods for inhibiting, reducing, preventing or controlling dental caries formation. Electrolyzed water having an oxidation-reduction potential from about −150 to 0 mV measured against a platinum electrode, also known as “Trim water” can reduce, inhibit, prevent, or control caries in part by neutralizing dental plaque acid by increasing the pH in the area surrounding dental plaque. One embodiment provides contacting a tooth with electrolyzed water, typically after the tooth has been challenged with a sugar, to neutralize dental plaque acid and thereby reduce the formation of dental caries. Electrolyzed water and compositions comprising electrolyzed water can be formulated as palatable beverages, gels, candy, or other foodstuffs that are safely digested by individuals such as young children.

### CROSS REFERENCE TO RELATED APPLICATION

This application claims benefit of priority to provisional patent application serial No. 60/340,769 entitled “The Effect of a Buffer on Acidogenesis of Plaque” filed on Dec. 11, 2001.

### BACKGROUND OF THE INVENTION

Dental caries occur in teeth where microbial plaques exists. Dental plaque is a soft deposit that accumulates on the teeth. Plaque can be defined as a complex microbial community, with greater than 10¹⁰ bacteria per milligram. It has been estimated that as many as 400 distinct bacterial species may be found in plaque. In addition to the bacterial cells, plaque contains a small number of epithelial cells, leukocytes, and macrophages. The cells are contained within an extracellular matrix, which is formed from bacterial products and saliva. The extracellular matrix contains protein, polysaccharide and lipids. Streptococcus sanguis, Streptococcus mutans, and Actinomyces viscosus are examples of primary microbial colonizers of dental plaque. Secondary colonizers include Gram-negative species such as Fusobacterium nucleatum, Prevotella intermedia, and Capnocytophaga species. Other microbial colonizers include Porphyromonas gingivalis, Campylobacter rectus, Eikenella corrodens, Actinobacillus actinomycetemcomitans, and the oral spirochetes (Treponema species).

Fermentable carbohydrates such as sugars in the diet are metabolized to acids such as lactic acid by plaque bacteria causing a pH change at the tooth surface. If the pH is sufficiently acidic and is not neutralized, the tooth, which is comprised mainly of calcium phosphate crystals such as hydroxylapatite will dissolve or decalcify producing a carious lesion. A pH at or below 5.5 is generally accepted as the threshold at which decalcification can proceed.

After a single sugar challenge, some surface decalcification will take place, but the damage will be reversed (recalcification) if the acid is neutralized within a short period of time after the sugar challenge (within 40 to 60 minutes). This neutralization, or buffering, takes place naturally by ammonia-producing bacteria in plaque. If sugar challenges are frequent, such as with a child who eats sweet snacks frequently during the day, neutralization of plaque generated acids and recalcification will not take place. Cavitation of the tooth will result if the latter process continues for a few months. Frequent sugar challenges also can cause the microbial composition of plaque to shift toward one which is more acidogenic and aciduric.

Prevention of the above processes would best be accomplished by limiting the dietary intake of sugar. This unfortunately is not possible with most children. Another approach would be enhancement of plaque neutralization after sugar challenges. This has been attempted experimentally by having subjects chew sugar-free chewing gum after sweet snacks, which theoretically stimulates saliva flow and thereby increases salivary bicarbonate concentrations, the main buffer in saliva. The increased saliva flow and the chewing action also helps clear the oral sugar concentrations. Reduction of dental caries has been reported using this technique (Scheinin, Acta Odont Scan 33:267, 1975).

Another approach would be to have subjects rinse their mouths with a buffering agent after a sugar snack. Although this has been tried experimentally in vitro (Macpherson, J Perio Res 26:395, 1991) it has not been developed into commercially available products. Reasons for this are not clear, but perhaps they would contain unsafe substances, which may be swallowed by young children, the prime beneficiaries of such a product.

Thus, there is a need for compositions and methods to reduce, inhibit, or prevent dental caries.

There is another need for beverages that reduce, inhibit, or prevent dental caries.

There is still another need for compositions and methods for controlling dental plaque acid production.

### SUMMARY OF THE INVENTION

The present invention provides compositions and methods for inhibiting, reducing, preventing or controlling dental caries, for example by neutralizing dental plaque acid. Electrolyzed water having an oxidation-reduction potential from about −150 to 0 mV measured against a platinum electrode, also known as “Trim water” can reduce, inhibit, neutralize or control dental plaque acid, for example by increasing the pH in the area surrounding the dental plaque. Other aspects include the control, reduction, prevention of the formation of dental plaque acid, for example by controlling, preventing, inhibiting, or reducing the growth of microorganisms that produce acid in the mouth. One embodiment provides contacting a tooth with electrolyzed water, typically after the tooth has been challenged with a sugar, to neutralize dental plaque acid and thereby reduce the formation of dental caries. Electrolyzed water and compositions comprising electrolyzed water can be formulated as palatable beverages, gels, candy, or other foodstuffs that are safely digested by individuals such as young children.

Thus, another aspect of the present invention provides a solution, for example an aqueous solution comprising electrolyzed water having an oxidation-reduction potential from about −150 to 0 mV. The solution can be a beverage which can be optionally sweetened, preferably with an artificial sweetener.

Still another aspect of the invention provides a dentifrice comprising electrolyzed water having an oxidation-reduction potential from about −150 to 0 mV. The dentifrice can be in the form of a paste, gel, film, rinse, mouth wash or solution. For example, tooth paste or a gel can be formulated to comprise an effective amount of electrolyzed water having an oxidation-reduction potential from about −150 to 0 mV. An effective amount of electrolyzed water having an oxidation-reduction potential from about −150 to 0 mV means an amount that increases the pH in an area of dental plaque to more than 5.5.

Another aspect of the invention provides a method of preventing or treating dental caries by contacting a tooth with a solution comprising electrolyzed water, wherein the electrolyzed water has an oxidization-reduction potential in the range of −150 to 0 mV. Contacting the tooth with the electrolyzed water solution cause the pH in the area in contact with the solution to increase, for example to a pH greater than 5.5. Reducing, neutralizing, or inhibiting acid formation by plaque helps control, reduce, or prevent dental caries.

The Trim water may be placed into a cariostatic formulation to be administered near the tooth. The Trim water may be acidic or alkaline. The time of administration may be for about one minute near the location of the teeth. For example, a mouthwash rinse may be rinsed for about 1 minute. Preferably, the mouth rinse or beverage of the invention results in the maintenance of pH above 5.5 near the tooth.

These and other aspects of the invention will be more fully understood from the following description of the invention, the referenced drawings attached hereto and the claims appended hereto.

### BRIEF DESCRIPTION OF THE DRAWINGS

The present invention will become more fully understood from the detailed description given below, and the accompanying drawings which are given by way of illustration only, and thus are not limitative of the present invention.

FIGS. 1A-B show the pH response of Streptococcus mutans in plain water or Trim water containing 5% sucrose.

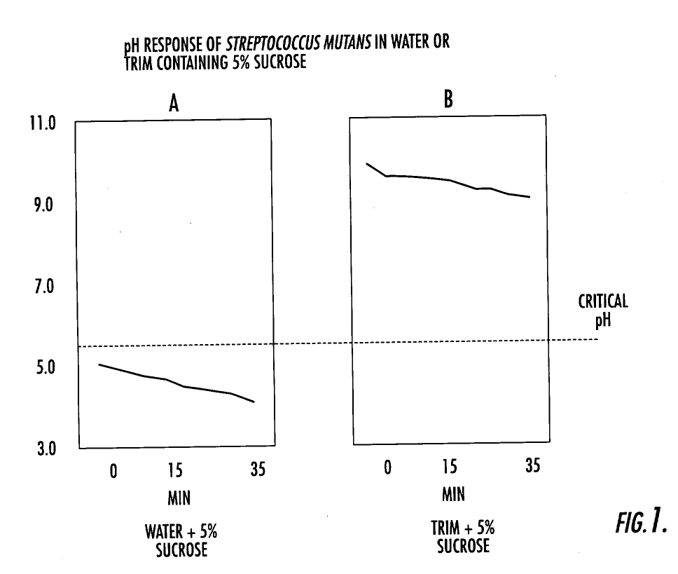

FIGS. 2A-B show the pH response of tooth #26 after a one-minute rinse with water.

FIGS. 3A-B show the pH response of tooth #26 after a one-minute rinse with Trim water.

FIGS. 4A-B show the pH response of tooth #27 after a one-minute rinse with water.

FIGS. 5A-B show the pH response of tooth #27 after a one-minute rinse with Trim water.

FIGS. 6A-B show the pH response of tooth #28 after a one-minute rinse with water.

FIGS. 7A-B show the pH response of tooth #28 after a one-minute rinse with Trim water.

FIGS. 8A-B show the pH response of tooth #29 after a one-minute rinse with water.

FIGS. 9A-B show the pH response of tooth #29 after a one-minute rinse with Trim water.

FIGS. 10A-B show the pH response of tooth #26 in Trim water containing 5% sucrose.

FIGS. 11A-B show the pH response of tooth #27 in Trim water containing 5% sucrose.

FIGS. 12A-B show the pH response of tooth #28 in Trim water containing 5% sucrose.

FIGS. 13A-B show the pH response of tooth #29 in Trim water containing 5% sucrose.

FIGS. 14A-L show sequential radiograph of teeth exposed to Streptococcus mutans and Lactobacilius casei.

### DETAILED DESCRIPTION OF THE INVENTION

The formation of dental caries is a widespread oral disease due in part to acids formed by dental plaque when foods, particularly foods containing sugars, are consumed by a host. Embodiments of the present invention provide compositions and methods for reducing, inhibiting, controlling, or preventing dental caries by contacting microorganisms that produce acids in oral cavities with electrolyzed water, for example electrolyzed water having an oxidation-reduction potential from about −150 to 0 mV measured against a platinum electrode, also known as “Trim water.” The growth of microorganisms in dental plaques can be controlled, reduced, or inhibited by applying Trim water to the dental plaques, for example using a mouth rinse, paste, gel, or cream comprising Trim water. Thus, one embodiment of the present invention provides a dentifrice comprising electrolyzed water having an oxidation-reduction potential from about −150 to 0 mV measured against a platinum electrode, also known as “Trim water.”

Definitions

As used herein, “Trim” and “Trim water” are used interchangeably. Furthermore, Trim water is described in U.S. Pat. No. 5,938,915, which is incorporated herein by reference in its entirety in the description of how Trim water that is either acidic or alkaline, are produced. Trim water includes electrolyzed water having a reduction-oxidation potential of about 150 to about 0 mV.

The term “host” means an organism such as a mammal, in particular a human, having at least one tooth.

The term “cariostatic agent” means a substance the treats, inhibits, prevents, or controls dental caries, for example and not limitation, fluoride.

The term “sweetener” means a natural or artificial substance that imparts or simulates the taste of sugar. Exemplary sweeteners include but are not limited to: acesulfame-potassium, aspartame, corn syrup, hydrogenated starch hydrolysates, isomalt, lactitol, maltitol, maltitol syrup, mannito, sorbitol, sorbitol syrup, sucralose, saccharine, thaumatin, and xylitol.

The term “dentifrice” means a powder, paste, or liquid for cleaning the teeth.

Production of Trim Water

Trim water is produced by electrolyzing water. In particular, Trim water includes electrolyzed water obtained from or near a cathode used in the electrolysis of the water. Electrolyzed water is rich in electrons (e⁻) and protons (H⁺). Rich or enriched in electrons and protons means having more electrons and protons than water not subjected to electrolysis, for example tap water. U.S. Pat. No. 5,938,915 discloses an apparatus and method for producing Trim water and is incorporated by reference herein in its entirety. Briefly, an electrolytic water treatment apparatus (HD-30) can be used which includes a cathode chamber with a cathode and an anode chamber with an anode. The cathode chamber can be separated from anode chamber by a diaphragm. A cathode liquid outlet pipe from which cathode liquid is drawn out is connected to cathode chamber. A drain pipe for discharging anode water is connected to anode chamber. Respective feed pipes are connected to cathode and anode chambers so that raw water optionally including at least one metal ion, for example sodium, potassium, magnesium, and calcium ions, such as tap water, ground water, or water from a well. Raw water means water that has not been treated.

Raw water such as tap water, ground water, or well water is supplied to a cathode chamber and an anode chamber. A current within the range of 0.16 mA/cm² to about 3.2 mA/cm² per each pair of electrodes and one diaphragm is applied across the cathode electrode and the anode electrode for at least 0.5 second and not more than 5 seconds at room temperature (18° C. to about 22° C.) to electrolyze the raw water. Exemplary electrolyzed water is obtained having a pH from about 8.0 to about 10.5, and an oxidation-reduction potential in the range of about −150 to about 0 mV. The oxidation-reduction potential (ORP) is measured against a platinum (Pt) electrode by an ORP measuring apparatus (RM-12P) of Toa Denpa Co. Ltd.

Embodiments

One embodiment of the present invention provides a beverage comprising electrolyzed water, for example electrolyzed water having an oxidation-reduction potential in the range of about −150 to about 0 mV. The electrolyzed water can have a pH in the range of about 8.0 to about 10.5. Exemplary beverages comprise from about 1 percent to about 90 percent electrolyzed water or Trim water. Typical beverages comprise an effective amount of electrolyzed water or Trim water, wherein an effective amount means an amount of electrolyzed water or Trim water that increases the pH of the area of interest, for example the surface of a tooth, the surface of a dental plaque, or the mouth, to greater than 5.5. The increase in pH can last from about 1 to about 120 minutes, typically at least about 25 minutes. Embodiments of the present invention can be used after a host has consumed foodstuffs containing sugars or other substances that can be converted to acids by microorganisms within the mouth. After consumption typically means within an hour of consumption, more typically within thirty minutes of consumption. Embodiments of the present invention increase the pH of an area of the mouth, including a tooth surface, and thereby control, prevent, inhibit or reduce the formation of dental caries in part by neutralizing acids that contribute to the disease.

The production of beverages is known in the art. Exemplary beverages can comprise additives, thickeners, flavor enhancers, emulsifiers, preservatives, coloring agents, sweeteners, vitamins, proteins, cofactors, carbohydrates, or carbonation. Electrolyzed water may be used as an additive to other beverages such as fruit juice, milk, or other drinks. Sweeteners can comprise less than 20 percent of the beverage, typically less than 10 percent, more typically less than 5 percent.

Another embodiment provides a dentifrice comprising electrolyzed water, for example electrolyzed water having an oxidation-reduction potential from about 0 to about −150 mV. The dentifrice can be formulated as a tooth paste, powder, mouth rinse or wash, gel, foam, or spray. Exemplary dentifrices comprise amounts of electrolyzed water sufficient to neutralize acid produced by microorganisms of the mouth, particularly microorganisms of the mouth. Exemplary microorganisms include but are not limited to Streptococcus sanguis, Streptococcus mutans, Actinomyces viscosus, Fusobacterium nucleatum, Prevotella intermedia, Capnocytophaga species, Porphyromonas gingivalis, Campylobacter rectus, Eikenella corrodens, Actinobacillus actinomycetemcomitans, Lactobacilius casei, and the oral spirochetes (Treponema species). The dentifrice can also comprise other agents, for example cariostatic agents such as fluorine, alcohol or the like. When applied to a dental plaque, tooth surface, or area or the mouth, the dentifrices of the present invention can increase the pH of the area of application, for example the dental plaque area and areas immediately adjacent to the dental plaque, to a pH of greater than 5.5. Typically, the increase in pH can remain above 5.5 from about 1 to about 120 minutes, more typically at least about 25 minutes.

Still another embodiment provides a method of preventing tooth decay. The method includes contacting a tooth with a solution comprising electrolyzed water, wherein the electrolyzed water has an oxidization-reduction potential in the range of −150 to 0 mV. Contacting the tooth surface with the compositions disclosed herein increases the pH of the tooth surface to greater than 5.5 for about 1 to 120 minutes, typically for at least about 25 minutes. Thus, the increase in pH neutralizes acids produced by microorganisms, particular microorganisms in dental plaque, and thereby prevents, reduces, or controls tooth decay.

Yet another embodiment provides a method of inhibiting, reducing, preventing, or controlling the growth of oral microorganisms, the method including contacting microorganisms in an oral cavity with a solution comprising electrolyzed water, wherein the electrolyzed water has an oxidization-reduction potential in the range of −150 to 0 mV. Microorganisms of the mouth include bacteria and fungi. Exemplary bacteria include but are not limited to Streptococcus sanguis, Streptococcus mutans, Actinomyces viscosus, Fusobacterium nucleatum, Prevotella intermedia, Capnocytophaga species, Porphyromonas gingivalis, Campylobacter rectus, Eikenella corrodens, Actinobacillus actinomycetemcomitans, and the oral spirochetes (Treponema species). Exemplary fungi include but are not limited to yeast such as Candida albicans. Electrolyzed water can increase the pH to greater than 5.5 in the area of the microorganisms for about 1 to about 120 minutes, typically at least about 25 minutes. Changes in pH interfere and can prevent the growth of microorganisms. Additionally, the increase concentration of electrons and or protons in the electrolyzed water can interfere, inhibit, or prevent the growth of microorganisms.

Experiments were designed to test anti-cariogenic potential of Trim as a 1) post sugar rinse, 2) base for a sweet beverage and 3) carostatic agent for active carious lesions.

Since in vivo rinsing will expose Trim water to human saliva which contains a bicarbonate buffering system, the pH of Trim water was measured after mouth rinsing for different period of time (Table 1). A 3-minute rinse with either acidic or alkaline Trim water showed salivary buffering to change the pH from 11.2 (alkaline) or 2.94 (acidic) to between 6 and 7. With shorter rinse of up to one minute, alkaline Trim remained above pH 9 even when sucrose was present.

Using caries model experiments the following advantage of the inventive method are seen.

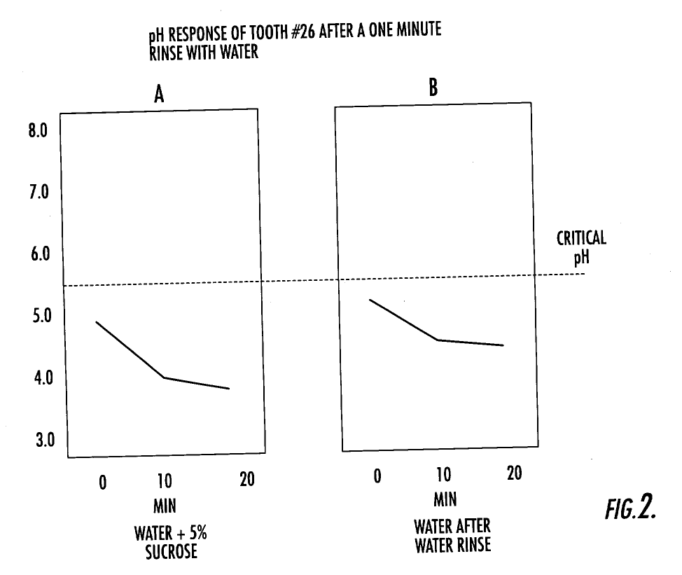

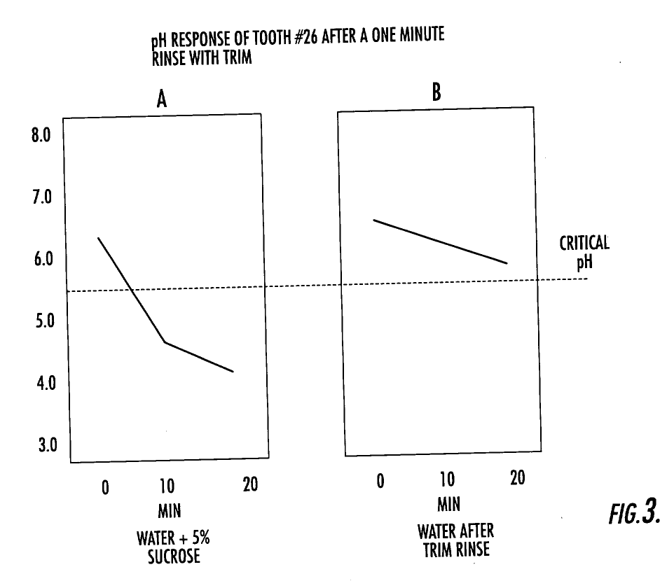

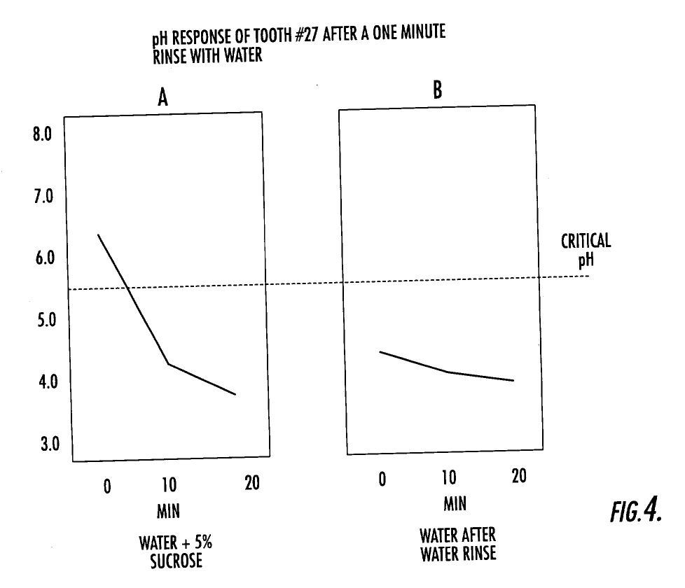

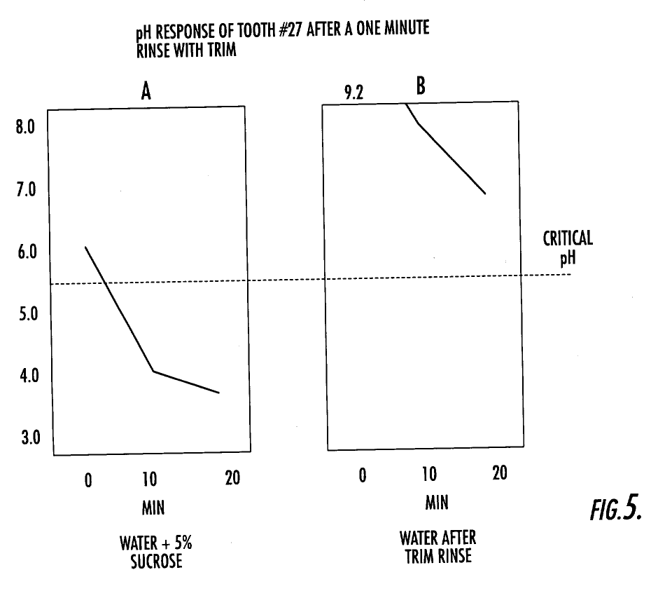

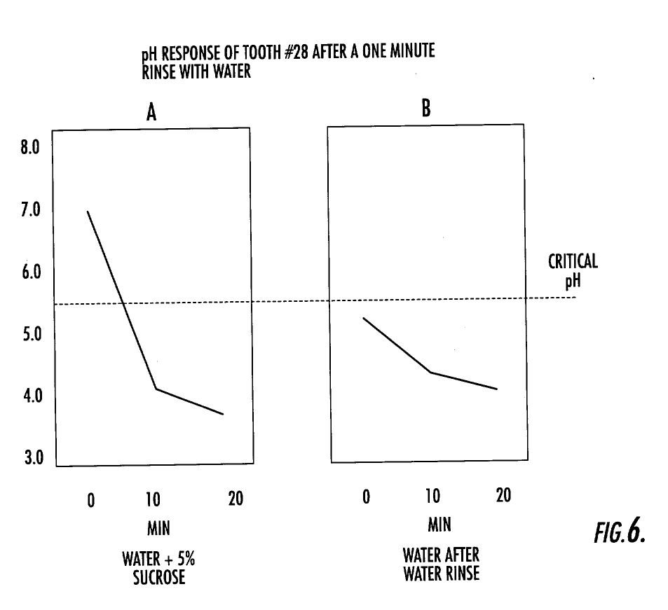

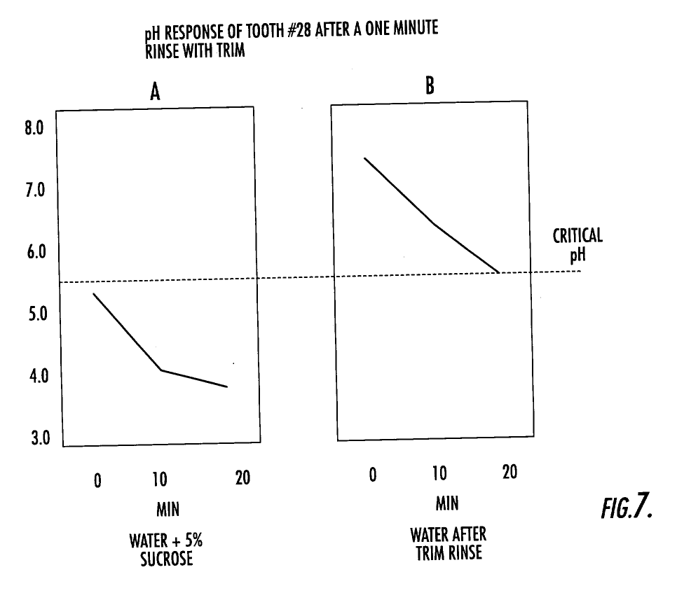

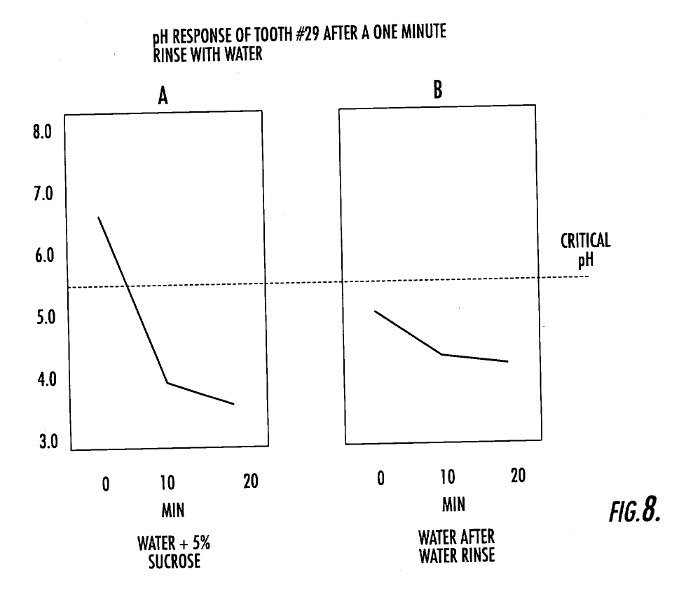

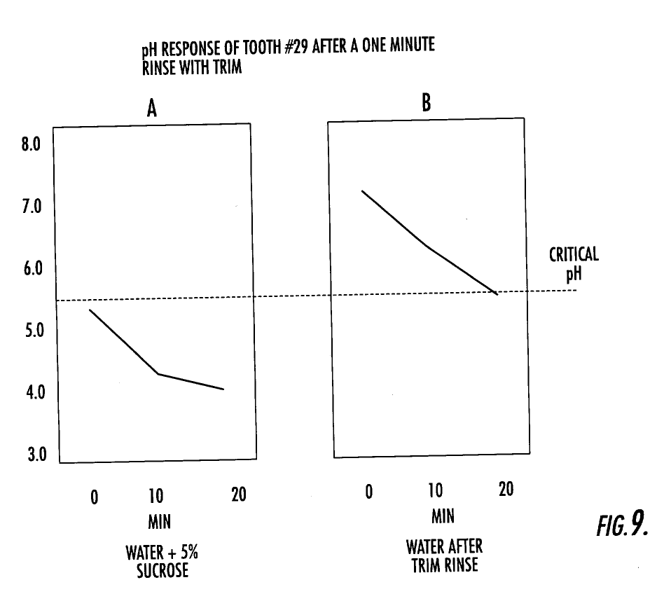

A 1-minute rinse with alkaline Trim water produced a rapid buffering of sucrose-exposed experimental cariogenic dental plaques. The pH remained above 5.5, which is the generally accepted pH when the enamel starts to decalcify, for at least 25 minutes. Normally, most sugar-containing snacks or beverage become cleared from the mouth within 25 minutes. Water rinsing did not provide an adequate anti-acid effect. (FIGS. 2-9).

Compared with water containing sucrose, Trim plus sucrose kept the pH above 5.5 for at least 25 minutes. When Trim plus sucrose was cleared from the plaques by a 1-minute water rinse, the pH continued to remain above 5.5 for an additional 25 minutes. Water plus sucrose failed to maintain the plaque pH within the protective range.

Trim rinsing did not greatly alter the pH within active experimental carious lesions which remained fairly constant at approximately pH 4.

Thus, frequent oral exposure to Trim in the form of a beverage or a mouth rinse shows caries-preventative effects, particularly if employed immediately after a sugar challenge. Immediately after a sugar challenge means within thirty minutes, more particularly within fifteen minutes of a sugar challenge.

Therefore, use of Trim as a base for a sweet beverage is preferable to water as a base. However, since there may be some residual cariogenic effects of sugar after Trim is cleared from the mouth, or buffered by saliva, Trim water combined with a sugar-free beverages is particularly advantageous. Such a beverage should provide a non-cariogenic, and produce an anti-caries effect since an alkaline pH enhances recalcification of teeth.

The following examples are offered by way of illustration of the present invention, and not by way of limitation.

### EXAMPLES

### Example 1

Effect of Trim Water on Streptococcus mutans Exposed to Sucrose.

The purpose of this experiment is to determine if Trim could reduce acidogenicity of S. mutans.

As a control, washed cells of Streptococcus mutans (GEM), the primary pathogen in human dental caries, was incubated in water containing 5% sucrose for 35 minutes. The pH changes were monitored with a pH meter. The same number of cells was also incubated in Trim water containing 5% sucrose for 35 minutes and the pH monitored.

Water containing 5% sucrose showed a decrease in pH from 5.02 to 4.06 during 35 minutes exposed to S. mutans. With Trim water containing 5% sucrose the pH decreased from 10.4 to 9.15. The later pH range was well within the safe range for tooth integrity. (Decalcification usually takes place below pH 5.5).

### Example 2

In vivo Mouth Rinse with Trim or Trim Plus Sucrose.

The purpose of this experiment is 1) to test the effect of oral exposure on the pH of Trim with and without sucrose and 2) to determine if Trim will buffer acidogensis of salivary bacteria for a prolonged period of time.

Trim containing 10% sucrose was rinsed in vivo for 3 minutes and then expectorated into a container, which was incubated at 37° C. for 110 minutes. The pH was monitored with a pH meter. A control solution with water containing 10% sucrose was examined similarly.

Trim was rinsed in vivo for 5, 10, 15 and 30 seconds. After each rinse it was expectorated into a container and the pH was measured. The same experiment was repeated with water.

Trim containing 10% sucrose was rinsed in vivo for 5, 15, 30 and 60 seconds. After each rinse it was expectorated into a container and the pH was measured. The same experiment was repeated with water.

A 3-minute rinse with Trim containing 10% sucrose in vivo caused a pH drop from 10.33 (original pH of Trim plus sucrose) to 6.25. After incubation of the expectorated rinse containing saliva for 110 minutes at 37° C., the pH decreased to 5.59.

The same experiment with water plus 10% sucrose resulted in a pH change from 5.61 to 6.45 after a 3-minute rinse in vivo. During the 110-minute incubation of the expectorated rinse, the pH dropped from 6.45 to 5.95.

The same experiment repeated with acidic Trim changed from a pH of 2.94 to 5.45 after a 3 minute rinse and to 5.53 after a 100 minute incubation of the expectorated mouthrinse.

These data sow strong buffering potential of saliva when Trim is kept in the mouth for 3 minutes. When shorter rinses (up to 1 minute) with trim were employed, the pH remained high.

TABLE 1
In Vivo Rinses with 2O ml of Trim or Water Solutions

| | | Water + | Acidic Trim + | | |
|---|---|---|---|---|---|
| | Trim + 10% | 10% sucrose | 10% sucrose | Trim | Water |
| Rinse time | sucrose pH | pH | pH | pH | pH |
| Original | 10.33 | 7.38 | 2.94 | 11.20 | 6.96 |
| 5 sec | 9.54 | 6.62 | 10.34 | 6.87 | |
| 10 sec | 10.24 | 6.78 | | | |
| 15 sec | (6.98) | 6.36 | 9.81 | 6.70 | |
| 30 sec | 9.04 | 6.19 | 9.27 | 6.63 | |
| 60 sec | 7.33 | 6.06 | | | |
| 3 min | 6.51 | 6.25 | 5.45 | | |
| Post | | | | | |
| Rinse | | | | | |
| 5 min | 5.45 | .21 | 5.45 | | |
| 10 min | 6.53 | 6.30 | 5.46 | | |
| 15 min | 6.49 | 6.27 | 5.48 | | |
| 20 min | 6.55 | 6.31 | 5.49 | | |
| 30 min | 6.55 | 6.31 | 5.49 | | |
| 35 min | 6.48 | 6.25 | 5.51 | | |
| 50 min | 6.36 | 6.19 | 5.52 | | |
| 110 min | 5.95 | 5.59 | 5.53 | | |

### Example 3

In Vitro Dental Caries Model

According to a Dentinal Caries Model System (Minah, Pediat. Dent 20:345, 1998), crowns of extracted in tact primary teeth were mounted in acrylic bases shaped to fit a holding jig in a digital radiograph apparatus, which permitted accurate positioning for sequential exposures. After enamel was covered with a layer of cold-cure acrylic, the dentin was exposed by circular openings 1.0 mm in diameter made with a #330 rotating dental bur. Dental caries-like lesions were induced by exposure for 6 weeks to Streptococcus mutans GEM, a biotype I clinical isolate (S. m), and Lactobacilius casei (ATCC 11578) (L.c.) according to the following protocol: mounted teeth were placed in brain Heart Infusion broth (BHI; Difco, Detroit, Mich.) containing 5.0% sucrose w/v which was inoculated with S.m. The medium was changed daily and incubation conducted at 37° C. for 7 days in air containing 10% CO₂. At day 8, mounted teeth were placed in MRS broth (Difco) containing 5.0% sucrose which was inoculated with L.c. This medium was alternated daily with a solution of 0.85% NaCl (normal saline) and 5.0% w/v sucrose. Incubation proceeded for an additional 97 days. Incremental caries progression was evaluated by sequential radiography. Spot microbial culturing of lesions was conducted.

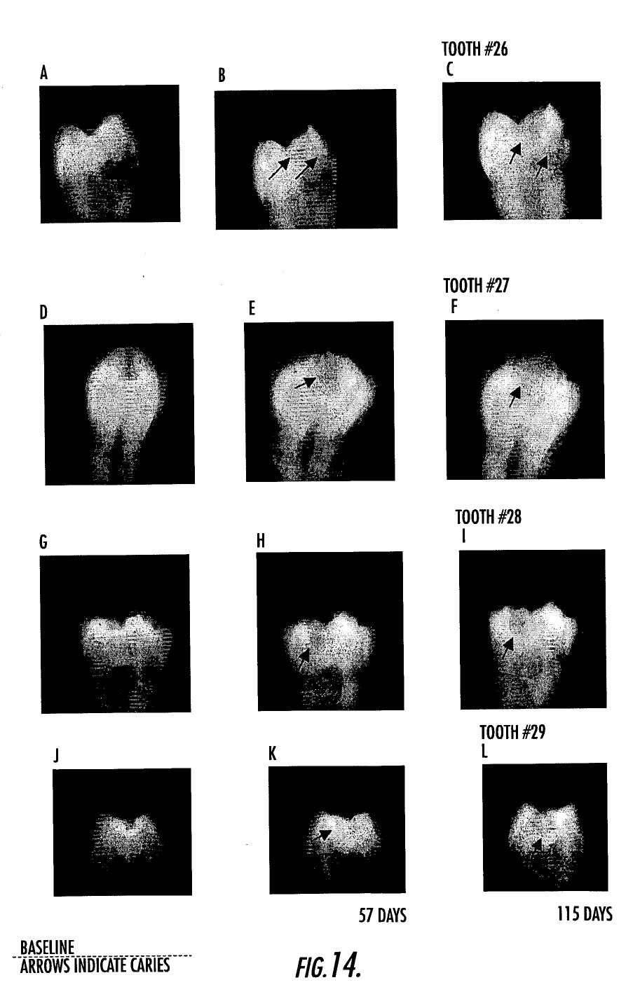

Teeth were incubated with pure cultures of cariogenic bacteria for 105 days. Dentinal-like carious lesions appeared in the teeth and progressed with time (FIGS. 14A to 14L).

### Example 4

Effect of a 1-Minute Rinse with Trim or Water on Acidogenesis of Experimental Dental Plaque (Indirect Plaque pH Measurements) See FIGS. 2-9.

The purpose of this experiment is to simulate the effect on acidogenesis of plaque following a 1-minute Trim rinse.

Mounted teeth containing carigenic plaques were removed from the incubation medium and rinsed in 0.85% saline solution (normal saline) to remove substrates and allow glycolysis to cease. Mounted teeth were then placed in 2 ml of water with 5% sucrose for 25 minutes at 37° C. This procedure simulated a typical dietary exposure to a cariogenic substrate. During this time pH of the water was measured by a pH meter (Orion 420 A, Fisher Scientific) with a Ross combination microelectrode (Orion, Fisher). Teeth were then be immersed for 1 min in ether Trim (test solution) or water (control solution) after which they were placed in 2 ml of water and the pH was monitored for additional 25 minutes.

Teeth exposed to 5% sucrose in water for 25 minutes decreased the plaque pH from approximately 6.5 to 4.00. After a one minute water rinse the pH returned to about 5.00 and dropped to 4.00 after an additional 25 minutes of incubation.

After a 1 minute Trim rinse, the pH rose to roughly 7.5 and then dropped to about 5.5 after an additional 25 minute incubation.

Trim appeared to have a protective effect on cariogenic plaque after a 1 minute rinse since acidity was immediately raised to a safe level and it remained above the critical pH of 5.5 for up to 25 minutes.

### Example 5

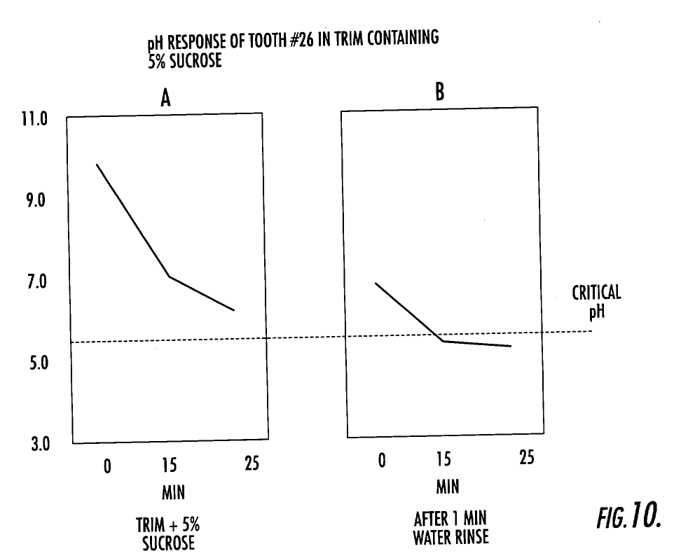

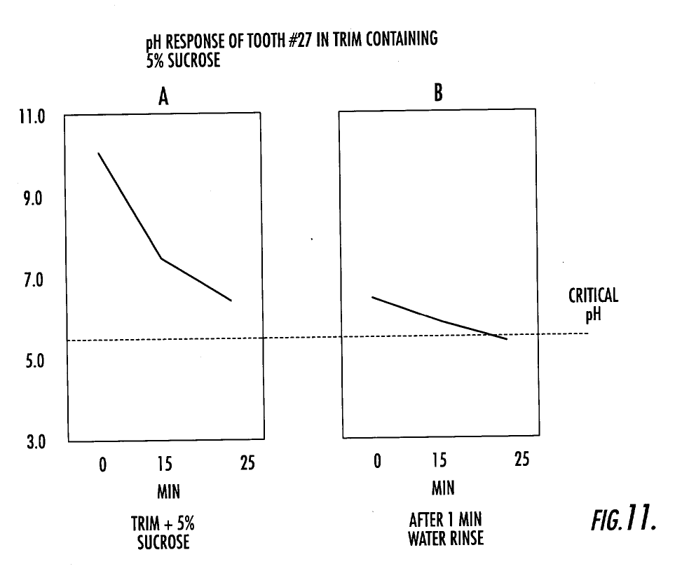

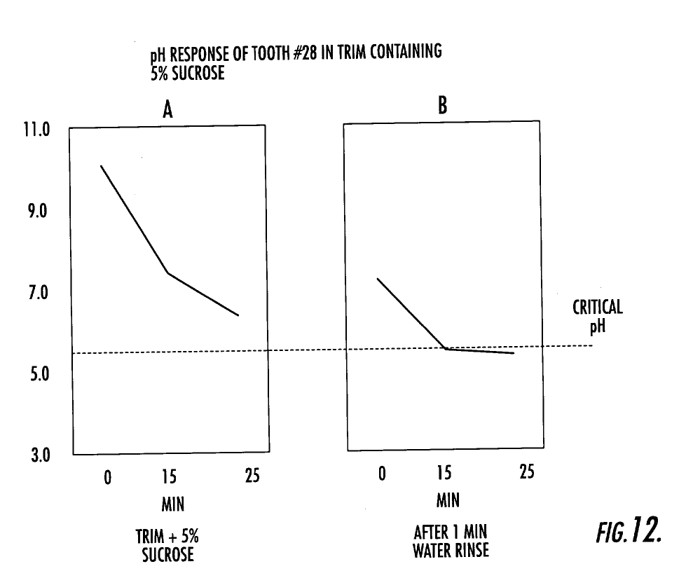

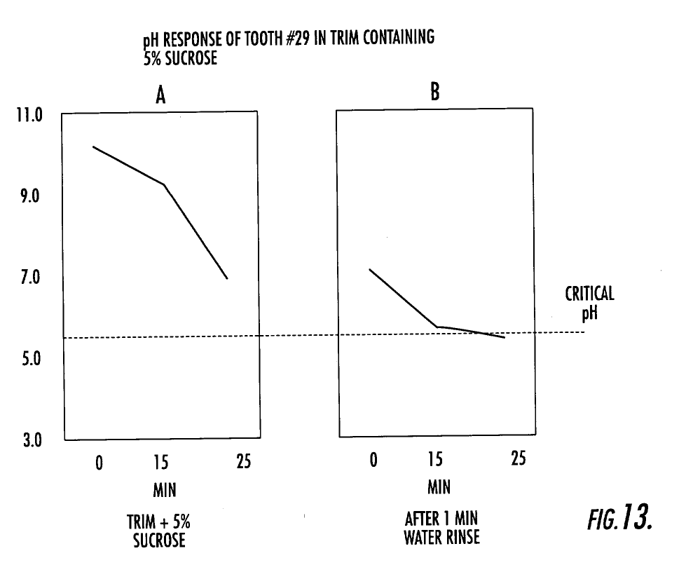

Effect of Trim or Water containing 5% Sucrose on Acidogenesis of Experimental Cariogenic Plaque (Indirect Plaque pH Measurements). See FIGS. 10-13.

The purpose of this experiment is to test plaque acidogenesis during exposure to Trim containing a cariogenic substrate.

The steps outlined in Example 2 above were repeated with the exception that Trim containing 5% sucrose replaced 5% sucrose in water during the first incubation. All teeth were then rinsed in water for 1 minute and the pH was followed for additional 25 minutes.

When Trim containing 5% sucrose was incubated with cariogenic plaques for 25 minutes the pH decreased from about 10.0 to 6.2. After a one minute water rinse the pH continued to drop to approximately 5.0 when incubated in water for additional 25 minutes.

These results indicate that if Trim were the base for a sweet drink, for example, pH of plaque would not decrease to a cariogenic level when the Trim drink is present in the mouth. Plaque acidogenesis would continue, however, after it is cleared by washing with water or perhaps, swallowing.

All of the references cited herein are incorporated by reference in their entirety.

Those skilled in the art will recognize, or be able to ascertain using no more than routine experimentation, may equivalents to the specific embodiments of the invention specifically described herein. Such equivalents are intended to be encompassed in the scope of the following claims.

What is claimed is:

1. A beverage comprising electrolyzed water, wherein the electrolyzed water has an oxidization-reduction potential in the range of about −150 to about 0 mV.

2. The beverage of claim 1, wherein the electrolyzed water comprises at least one metal ion.

3. The beverage of claim 2, wherein the metal ion is selected from the group consisting of sodium, potassium, magnesium, and calcium.

4. The beverage of claim 1, wherein the beverage increases the pH of dental plaque in contact with the beverage to greater than 5.5.

5. The beverage of claim 1, further comprising a sweetener.

6. The beverage of claim 5, wherein the sweetener is selected from the group consisting of acesulfame-potassium, corn syrup, dextrose, hydrogenated starch hydrolysates, fructose, glucose, isomalt, lactitol, maltitol, maltitol syrup, mannito, sorbitol, sorbitol syrup, sucrose, sucralose, thaumatin, and xylitol.

7. The beverage of claim 5, wherein the sweetener is an artificial sweetener.

8. The beverage of claim 7, wherein the artificial sweetener is selected from the group consisting of saccharine and aspartame.

9. A method for preventing tooth decay, the method comprising the step of:
contacting a tooth surface with a solution comprising electrolyzed water, wherein the electrolyzed water has an oxidization-reduction potential in the range of −150 to 0 mV.

10. The method of claim 9, wherein the solution contacts the tooth surface for at least one minute.

11. The method of claim 9, wherein the solution increases the pH of the tooth surface to more than 5.5.

12. A mouth rinse comprising electrolyzed water for decreasing the hydrogen ion concentration of an oral cavity, wherein the electrolyzed water has an oxidization-reduction potential in the range of −150 to 0 mV.

13. The mouth rinse of claim 12, further comprising a sweetener.

14. The mouth rinse of claim 13, wherein the sweetener is selected from the group consisting of acesulfame-potassium, corn syrup, dextrose, hydrogenated starch hydrolysates, fructose, glucose, isomalt, lactitol, maltitol, maltitol syrup, mannito, sorbitol, sorbitol syrup, sucrose, sucralose, thaumatin, and xylitol.

15. The mouth rinse of claim 13, wherein the sweetener is an artificial sweetener.

16. The beverage of claim 15, wherein the artificial sweetener is selected from the group consisting of saccharine and aspartame.

17. A cariostatic agent comprising electrolyzed water, wherein the electrolyzed water has an oxidization-reduction potential in the range of −1 50 to 0 mV.

18. A method for preventing tooth decay, the method comprising the step of:
increasing the pH of a tooth surface to greater than 5.5 by contacting the tooth surface with a solution comprising electrolyzed water, wherein the electrolyzed water has an oxidization-reduction potential in the range of −150 to 0 mV.

19. A dentifrice comprising electrolyzed water, wherein the electrolyzed water has an oxidization-reduction potential in the range of −150 to 0 mV.

20. A method of inhibiting the growth of oral microorganisms, the method comprising:
contacting microorganisms in an oral cavity with a solution comprising electrolyzed water, wherein the electrolyzed water has an oxidization-reduction potential in the range of −150 to 0 mV.

21. The method of claim 20, wherein the solution increases the pH in an area of the oral cavity to 5.5 or greater.

22. The method of claim 20, wherein the solution comprises a sweetener.

23. The beverage of claim 22, wherein the sweetener is selected from the group consisting of acesulfame-potassium, corn syrup, dextrose, hydrogenated starch hydrolysates, fructose, glucose, isomalt, lactitol, maltitol, maltitol syrup, mannito, sorbitol, sorbitol syrup, sucrose, sucralose, thaumatin, and xylitol.

24. The beverage of claim 22, wherein the sweetener is an artificial sweetener.

25. The beverage of claim 24, wherein the artificial sweetener is selected from the group consisting of saccharine and aspartame.

26. The method of claim 20, wherein the microorganisms comprise Streptococcus sanguis, Streptococcus mutans, Actinomyces viscosus, Fusobacterium nucleatum, Prevotella intermedia, Capnocytophaga species, Porphyromonas gingivalis, Campylobacter rectus, Eikenella corrodens, Actinobacillus actinomycetemcomitans, Lactobacilius casei, the oral spirochetes (Treponema species) and Candida albicans, or a combination thereof.

27. A solution comprising:
electrolyzed water, wherein the electrolyzed water has an oxidization-reduction potential in the range of −150 to 0 mV; and
a sweetener.

28. The solution of claim 27, further comprising a coloring agent.

29. The solution of claim 27, further comprising carbonation.
